(19) United States
(12) Patent Application Publication
(10) Pub. No.: US 2015/0057519 A1
(43) Pub. Date: Feb. 26, 2015

(54) SENSING SYSTEM, DEVICE AND METHODS FOR GASTROPARESIS MONITORING

(71) Applicant: GEMA MEDICAL LTD., Jerusalem (IL)

(72) Inventors: Matan Ben-David, Tel Aviv (IL); Moshe Rubin, Beit Yanai (IL); Raz Bar-On, Hadera (IL); Yuval Andorn, Tel Aviv (IL); Jacob Bortman, Rehovot (IL)

(73) Assignee: GEMA MEDICAL LTD., Jerusalem (IL)

(21) Appl. No.: 14/385,289

(22) PCT Filed: Mar. 14, 2013
(86) PCT No.: PCT/IL2013/050247

§ 371 Date: Sep. 15, 2014

Related U.S. Application Data
(60) Provisional application No. 61/611,026, filed on Mar. 15, 2012.

(51) Int. Cl. A61B 5/00; A61N 1/36; A61B 5/0492

(52) U.S. Cl. CPC A61B 5/4238; A61B 5/0492; A61B 5/6871; A61N 1/36007
USPC 600/373; 607/40

(57) ABSTRACT

An expandable apparatus and method configured for inserting into a mammalian stomach and adapted for sensing the activity of the stomach wall. The apparatus comprising: a) an inflatable balloon insert-able into the stomach, when deflated; and b) one or more sensors, mounted to the external surface of the balloon. The inflatable balloon is adapted to attach the one or more sensors to the stomach wall, when the balloon is fully inflated in the stomach; such that the sensors are immovably affixed to the stomach wall, sufficient to sense at least one of mechanical-activity and electric-activity, emanating from the stomach wall.

### FIELD OF INVENTION

The present invention relates to systems capable of measuring and detecting electrical and mechanical signals of the stomach and, more particularly means for diagnosis and treatment of motility disorders.

### BACKGROUND

The present invention discloses means for diagnosis of gastric dysmotility. The following prior art is believed to be the current status of the art:

US patent no. 2010113939 reveals a balloon-like catheter adapted for measuring pressure in the stomach. It describes different structures of a catheter, however it fails to disclose an apparatus adapted to measure electrical signals.

DE3523987 discloses a device for measuring the degree of denervation of the stomach. The device is an elastic balloon adapted to match the stomach wall and measures the PH level of the stomach. The invention does not disclose measuring the electrical signals of the stomach in order to assess the denervation of the stomach.

US patent no. 2005216040 discloses methods and apparatus for implantation into the walls of the stomach. Inflatable anchors with a connector between are used to pull the walls of the organ together, or to implant devices in the wall of the organ. The anchors can deliver an electrical signal to tissue when placed in contact with the tissue.

US patent no. 2007265598 discloses a balloon to be inserted into a patient stomach for the purpose of treating weight disorder. The invention includes an inflatable balloon with electrodes such as microelectrodes implanted under the esophagus endothelium; however, these electrodes are used to stimulate the stomach for controlling hunger, and not for detection of electrical movement.

US patent no. 2005215981 reveals a diagnosis catheter for an interstitial cystitis with an inflating balloon adapted to detect current in the bladder and apply electric current upon demand. The catheter comprises ring-like electrodes that extend continuously in a peripheral direction of the catheter body.

U.S. Pat. No. 4,721,115 relates to a diagnostic catheter adapted to detect cardiac output with an inflatable balloon. The catheter comprises an elongated plastic tubular member having a plurality of lumens running the length thereof and surface electrodes in the form of conductive rings mounted on the exterior surface of the catheter body.

US patent no. 2005049475 relates to a method for stimulating walls of hollow bodily systems, and also for measuring sensational or physical reactions towards such stimulation by introducing from an exteriorly accessible opening of a bodily hollow system a catheter into the hollow system. The catheter is provided with an inflatable balloon situated between a proximal end and a distal end of the catheter. The balloon is inflated until the balloon abuts an inner wall of the hollow system in order for the balloon and the catheter to be fixed in relation to the hollow system. The invention fails to disclose measuring physical parameters without an artificial stimulation.

Gastroparesis, also known as delayed gastric emptying, is a medical condition consisting of: a paresis (partial paralysis) of the stomach, resulting in food remaining in the stomach for a longer period of time than normal. Normally, the stomach contracts to move food down into the small intestine for digestion. The vagus nerve and the interstial cells of cajal control these contractions. Gastroparesis may occur when this system is damaged and the muscles of the stomach and intestines do not work normally. Food then moves slowly or stops moving through the digestive tract.

There is therefore, a long felt unmet need for better diagnosis of Gastroparesis and other gastric motility disorders such as functional dyspepsia, unexplained nausea and vomiting and GERD, and more particularly, methods and devices for detecting physical movement and motility of the stomach, preferably by minimally invasive simple electrical means.

### SUMMARY OF THE INVENTION

It is one object of the present invention to disclose an expandable apparatus 1 for inserting into a mammalian stomach and sensing the activity of the stomach wall 8, comprising:
a. an inflatable balloon 4 insert-able into said stomach, when deflated; and
b. one or more sensors 2, mounted to the external surface 7 of said balloon 4;
   wherein said inflatable balloon 4 is adapted to attach said one or more sensors 2 to said stomach wall 8, when said balloon 4 is fully inflated in said stomach; such that said sensors 2 are immovably affixed to said stomach wall 8, sufficient to sense at least one of: mechanical-activity and electric-activity, emanating from said stomach wall 8.

It is another object of the present invention to disclose the apparatus 1 as defined above, wherein said apparatus 1 is capable of measuring and detecting Gastroparesis.

It is another object of the present invention to disclose the apparatus 1 as defined above, wherein said sensors 2 are electrodes 6 selected from a group consisting of: heat, thermal or temperature electrode, mechanical electrode, electro-mechanical electrode, chemical electrode, gas electrode, electric current electrode, electric potential electrode, pressure electrode, strain electrode, acceleration electrode, piezo-electric electrode, and any combination thereof.

It is another object of the present invention to disclose the apparatus 1 as defined above, wherein said one or more sensors 2 are arranged in a non-contact multi-sensors mesh arrangement.

It is another object of the present invention to disclose the apparatus 1 as defined above, wherein said mesh arrangement is adapted for translating the stomach electrical information into a 3D or 2D isopotential map.

It is another object of the present invention to disclose the apparatus 1 as defined above, wherein said apparatus 1 further comprises at least one catheter 20 configured for at least one of: to control the inflation and deflation of said balloon 4; and for ablation procedure.

It is another object of the present invention to disclose the apparatus 1 as defined above, wherein said one or more sensors 2 comprises at least one hardware-device 12 adapted to save the data sensed by said one or more sensors 2.

It is another object of the present invention to disclose the apparatus 1 as defined above, wherein said sensors 2 are in communication with a processor 10; said communication is done by connecting said hardware-device 12 to said processor 10.

It is another object of the present invention to disclose the apparatus 1 as defined above, wherein said processor 10 is programmed to filter background electrical-noise from sensed electrical activity.

It is another object of the present invention to disclose the apparatus 1 as defined above, wherein said hardware-device 12 is mounted on either one of said one or more sensors 2.

It is another object of the present invention to disclose the apparatus 1 as defined above, wherein said communication between said processor 10 and at least one of: said sensors 2 and said hardware-device 12, are done via a communication element selected from a group comprising of: USB cable, serial cable, LAN, Bluetooth, Wi-Fi, any other element of physical or wireless connection, and any combination thereof.

It is another object of the present invention to disclose the apparatus 1 as defined above, wherein said inflatable balloon 4 is mounted with a plurality of said one or more sensors 2, configured to map said electric activity of said stomach.

It is another object of the present invention to disclose the apparatus 1 as defined above, wherein said inflatable balloon 4 is made of an elastic polymer.

It is another object of the present invention to disclose the apparatus 1 as defined above, wherein said inflatable balloon 4 has size and structure configured to bring said one or more sensors 2 in contact with stomach sections such as: cardia, fundus, body and Antrum.

It is another object of the present invention to disclose the apparatus 1 as defined above, wherein said inflatable balloon 4 has size and structure configured to bring said one or more sensors 2 in contact with the stomach mucosa layer.

It is another object of the present invention to disclose the apparatus 1 as defined above, wherein said apparatus 1 is capable of sensing physiological changes associated with food ingestion.

It is another object of the present invention to disclose the apparatus 1 as defined above, wherein said sensors 2 are fixedly attached to said balloon 4 via any known method for embedding a sensor to an expandable member.

It is another object of the present invention to disclose the apparatus 1 as defined above, wherein said sensors 2 are fixedly attached to said balloon 4 via thin semiconductors 13 bend-able on said balloon's external surface 7.

It is another object of the present invention to disclose the apparatus 1 as defined above, wherein said semiconductors 13 are sized such that they can be patterned in curves along the tip of said balloon 4.

It is another object of the present invention to disclose the apparatus 1 as defined above, wherein said one or more sensors 2 mounted on said semiconductor 13 are held together via springy connectors 14.

It is another object of the present invention to disclose the apparatus 1 as defined above, wherein said sensors 2 have shape and size selected from the group consisting of: circular shape, elliptic shape, disc shape, and any closed curved structure, such that no piercing or any other injury or damage is preformed to said stomach wall 8.

It is another object of the present invention to disclose the apparatus 1 as defined above, wherein said sensors 2 are combined into multi-functional sensor-platforms for neutralizing the area of said stomach wall 8 which said sensors 2 are in contact with.

It is another object of the present invention to disclose the apparatus 1 as defined above, wherein said sensors 2 are attached to said external surface 7 of said inflatable balloon 4, in a manner which prevents detaching or slipping of said sensors 2 from said balloon 4.

It is another object of the present invention to disclose the apparatus 1 as defined above, wherein said one or more electrodes 6 are adapted for stimulating electrical-pulses in order to provide therapy or to alleviate symptoms of obesity, bulimia or eating disorders.

It is another object of the present invention to disclose the apparatus 1 as defined above, wherein said balloon 4 comprises a tube 3, connected on one end 5 to said balloon 4, configured to inflate and deflate said balloon 4.

It is another object of the present invention to disclose the apparatus 1 as defined above, wherein said inflatable balloon 4 has a mesh-like configuration 40.

It is another object of the present invention to disclose the apparatus 1 as defined above, wherein said mesh-like configuration 40 comprises: interconnected inflatable tubular segments 50 enclosing at least one of: polygonal-spaces and oval-spaces, and provided with an open central hollow space 53 or partially closed central hollow space 54.

It is another object of the present invention to disclose the apparatus 1 as defined above, wherein said polygonal-spaces are selected from a group consisting of: triangle, square 51, diamond 52, rectangular, pentagon, hexagon, heptagon, octagon and any combination thereof.

It is another object of the present invention to disclose the apparatus 1 as defined above, wherein said mesh-like configuration 40 is provided with at least one inflatable ring segment 55.

It is another object of the present invention to disclose the apparatus 1 as defined above, wherein said balloon 4 is embedded within a scaffolding-shield 60 and wherein said sensors 2 are connected to the outer surface 61 of said scaffolding-shield 60; such that said scaffolding-shield 60 immovably affixes said sensors 2 to said stomach wall 8, when said balloon 4 fully inflated.

It is another object of the present invention to disclose the apparatus 1 as defined above, wherein said scaffolding-shield 60 is made of a material selected from a group consisting of: polymers, metals, alloys, memory shapes alloys and any combination thereof.

It is another object of the present invention to disclose the apparatus 1 as defined above, wherein said sensors 2 are printed onto said balloon's 4 external surface 7, by three-dimensional (3D) printing techniques.

It is another object of the present invention to disclose the apparatus 1 as defined above, wherein said hardware-device 12 is printed onto said balloon's 4 external surface 7, adjacent to said sensors 2, by three-dimensional (3D) printing techniques.

It is another object of the present invention to disclose the apparatus 1 as defined above, wherein said semiconductors 13 are printed onto said balloon's 4 external surface 7, adjacent to said sensors 2, by three-dimensional (3D) printing techniques.

It is another object of the present invention to disclose a system 100 for detecting stomach electrical activity, comprising:
a. an inflatable balloon 4, adapted to be inserted into an organ stomach;
b. one or more electrodes 6, mounted on the external surface 7 of said balloon 4;
c. a tube 3, connected on one end 5 to said balloon 4, configured to inflate and deflate said balloon 4; and
d. a processor 10 in communication with said one or more electrodes 6;
   wherein said inflatable balloon 4 is adapted to bring said one or more electrodes 6 into substantially immobile contact with the stomach wall 8, when said balloon 4 is fully inflated in said stomach; said electrodes 6 are immovably affixed to said stomach wall 8 sufficient to sense an electric activity emanating from said stomach wall 8; further wherein, said activity sensed by said electrodes 6 is transmitted to said processor 10; said processor 10 is programmed to filter background electrical noise from said sensed electrical activity.

It is another object of the present invention to disclose the system 100 as defined above, wherein said system 100 is capable of measuring and detecting Gastroparesis.

It is another object of the present invention to disclose the system 100 as defined above, wherein said electrodes 6 are selected from a group consisting of: heat, thermal or temperature electrode, mechanical electrode, electro-mechanical electrode, chemical electrode, gas electrode, electric current electrode, electric potential electrode, pressure electrode, strain electrode, acceleration electrode, piezo-electric electrode, and any combination thereof.

It is another object of the present invention to disclose the system 100 as defined above, wherein said electrodes 6 are arranged in a non-contact multi-electrode mesh arrangement.

It is another object of the present invention to disclose the system 100 as defined above, wherein said mesh arrangement is adapted for translating the stomach electrical information into a 3D or 2D isopotential map.

It is another object of the present invention to disclose the system 100 as defined above, wherein said system 100 further comprises at least one catheter 20 configured for at least one of: to control the inflation and deflation of said balloon 4; and for ablation procedure.

It is another object of the present invention to disclose the system 100 as defined above, wherein said catheter 20 controls the inflation and deflation of said balloon 4.

It is another object of the present invention to disclose the system 100 as defined above, wherein said one more electrodes 6 comprise a hardware-device 12, adapted to save data sensed by said one or more electrodes 6.

It is another object of the present invention to disclose the system 100 as defined above, wherein said communication between electrodes 6 and said processor 10 is done by connecting said hardware-device 12 to said processor 10.

It is another object of the present invention to disclose the system 100 as defined above, wherein said hardware-device 12 is mounted on either one of said electrodes 6.

It is another object of the present invention to disclose the system 100 as defined above, wherein said communication between said processor 10 and at least one of: said electrodes 6 and said hardware-device 12, is done via a communication method selected from a group comprising of USB cable, serial cable, LAN, Bluetooth, Wi-Fi, any other method of physical or wireless connection, and a combination thereof.

It is another object of the present invention to disclose the system 100 as defined above, wherein said inflatable balloon 4 is mounted with a plurality of said electrodes 6, configured to map the electric activity of said stomach wall 8.

It is another object of the present invention to disclose the system 100 as defined above, wherein said inflatable balloon 4 is comprised of an elastic polymer.

It is another object of the present invention to disclose the system 100 as defined above, wherein said inflatable balloon 4 has size and structure configured to bring said electrodes 6 in contact with stomach sections such as: cardia, fundus, body and Antrum.

It is another object of the present invention to disclose the system 100 as defined above, wherein said inflatable balloon 4 has size and structure configured to bring said electrodes 6 in contact with the stomach mucosa layer.

It is another object of the present invention to disclose the system 100 as defined above, wherein said system 100 capable of sensing physiological changes associated with food ingestion.

It is another object of the present invention to disclose the system 100 as defined above, wherein said electrodes 6 are fixedly attached to said balloon 4 is done via any known method for embedding an electrode to an expandable member.

It is another object of the present invention to disclose the system 100 as defined above, wherein said electrodes 6 have shape and size selected from the group consisting of: circular shape, elliptic shape, disc shape, and any closed curved structure, such that no piercing or any other injury or damage is preformed to said stomach wall 8.

It is another object of the present invention to disclose the system 100 as defined above, wherein said electrodes 6 are combined into multi-functional electrode-platforms for neutralizing the area of the stomach wall 8, which said electrodes 6 are in contact with.

It is another object of the present invention to disclose the system 100 as defined above, wherein said electrodes 6 are attached to the external surface 7 of said inflatable balloon 4, in a manner which prevents detaching or slipping of said electrodes 6 from said balloon 4.

It is another object of the present invention to disclose the system 100 as defined above, wherein said electrodes 6 are fixedly attached to said balloon 4 via thin semiconductors 13 that could bend on said balloon's external surface 7.

It is another object of the present invention to disclose the system 100 as defined above, wherein said semiconductors 13 are sized such that they can be patterned in curves along the balloon's 4 tip.

It is another object of the present invention to disclose the system 100 as defined above, wherein said electrodes 6, mounted on said semiconductor 13, are held together via springy connectors 14.

It is another object of the present invention to disclose the system 100 as defined above, wherein said system 100 is adapted for stimulating electrical pulses in order to provide therapy or to alleviate symptoms of obesity, bulimia or eating disorders.

It is another object of the present invention to disclose the system 100 as defined above, wherein said inflatable balloon 4 has a mesh-like configuration 40.

It is another object of the present invention to disclose the system 100 as defined above, wherein said mesh-like configuration 40 comprising: interconnected inflatable tubular segments 50 enclosing at least one of: polygonal-spaces and oval-spaces, and provided with an open central hollow space 53 or partially closed central hollow space 54.

It is another object of the present invention to disclose the system 100 as defined above, wherein said polygonal-spaces are selected from a group consisting of: triangle, square 51, diamond 52, rectangular, pentagon, hexagon, heptagon, octagon and any combination thereof.

It is another object of the present invention to disclose the system 100 as defined above, wherein said mesh-like configuration 40 is provided with at least one inflatable ring segment 55.

It is another object of the present invention to disclose the system 100 as defined above, wherein said balloon 4 is embedded within a scaffolding-shield 60 and wherein said sensors 2 are connected to the outer surface 61 of said scaffolding-shield 60; such that said scaffolding-shield 60 immovably affixes said sensors 2 to said stomach wall 8, when said balloon 4 fully inflated.

It is another object of the present invention to disclose the system 100 as defined above, wherein said scaffolding-shield 60 is made of a material selected from a group consisting of: polymers, metals, alloys, memory shapes alloys and any combination thereof.

It is another object of the present invention to disclose the system 100 as defined above, wherein said sensors 2 are printed onto said balloon's 4 external surface 7, by three-dimensional (3D) printing techniques.

It is another object of the present invention to disclose the system 100 as defined above, wherein said hardware-device 12 is printed onto said balloon's 4 external surface 7, adjacent to said sensors 2, by three-dimensional (3D) printing techniques.

It is another object of the present invention to disclose the system 100 as defined above, wherein said semiconductors 13 are printed onto said balloon's 4 external surface 7, adjacent to said sensors 2, by three-dimensional (3D) printing techniques.

It is another object of the present invention to disclose a method for detecting stomach activity, said method comprising steps of:
a. providing an expandable apparatus 1 comprising:
   i. an inflatable balloon 4 adapted to be inserted into a mammalian stomach;
   ii. one or more sensors 2; said sensors 2 are mounted on the external surface 7 of said balloon 4; and
   iii. a tube 3 connected on one end 5 to said balloon 4, adapted for inflating and deflating said balloon 4;
b. inserting said apparatus 1 comprising said balloon 4 in its deflated configuration into a patient hollow stomach system;
c. orienting said inflatable balloon 4 adjacent to stomach wall 8, thereby contacting said sensors 2 with said stomach wall 8;
d. inflating said inflatable balloon 4 to an immovably affixed contact with the stomach wall 8;
e. sensing at least one of: mechanical-activity and electric-activity, by said one or more sensors 2;
f. communicating said sensed electrical activity from said one or more sensors 2 to a processor 10, and;
g. filtering background electrical noise from said sensed electrical activity;
   wherein said inflatable balloon 4 is adapted for attaching said one or more sensors 2 to said stomach wall 8, when said balloon 4 is fully inflated in said stomach; thereby said sensors 2 are immovably affixed to said stomach wall 8, sufficient for sensing at least one of: mechanical-activity and electric-activity, emanating from said stomach wall 8.

It is another object of the present invention to disclose the method as defined above, wherein said method is capable of measuring and detecting Gastroparesis.

It is another object of the present invention to disclose the method as defined above, wherein said sensors 2 are adapted for sensing physiological change of said stomach.

It is another object of the present invention to disclose the method as defined above, further comprising a step of accumulating and analyzing the data detected by said sensors 2 in order to filter said data corresponding to stomach movement.

It is another object of the present invention to disclose the method as defined above, further comprising a step of transmitting the activity sensed by said sensors 2 to said processor 10.

It is another object of the present invention to disclose the method as defined above, wherein said sensors 2 are electrodes 6 selected from a group consisting of: heat, thermal or temperature electrode, mechanical electrode, electro-mechanical electrode, chemical electrode, gas electrode, electric current electrode, electric potential electrode, pressure electrode, strain electrode, acceleration electrode, piezo-electric electrode and any combination thereof.

It is another object of the present invention to disclose the method as defined above, wherein said one or more sensors 2 are arranged in a non-contact multi-sensor mesh arrangement.

It is another object of the present invention to disclose the method as defined above, wherein said mesh arrangement is adapted for translating the stomach electrical information into a 3D or 2D isopotential map.

It is another object of the present invention to disclose the method as defined above, wherein said tube 3 is a catheter 20.

It is another object of the present invention to disclose the method as defined above, wherein said catheter 20 configured to control at least one of: the inflation and deflation of said balloon 4 and for ablation procedure.

It is another object of the present invention to disclose the method as defined above, wherein said one or more sensors 2 comprise a hardware-device 12, adapted to save data sensed by said one or more sensors 2.

It is another object of the present invention to disclose the method as defined above, wherein said communication between sensors 2 and processor 10 is done by connecting said hardware-device 12 to said processor 10.

It is another object of the present invention to disclose the method as defined above, wherein said hardware-device 12 is mounted on either one of said one or more sensors 2.

It is another object of the present invention to disclose the method as defined above, wherein said communicating between said processor 10 and at least one of: said sensors 2 and said hardware-device 12, is done via a communication method selected from a group comprising of USB cable, serial cable, LAN, Bluetooth, Wi-Fi, any other method of physical or wireless connection, and a combination thereof.

It is another object of the present invention to disclose the method as defined above, wherein said inflatable balloon 4 is mounted with a plurality of said sensors 2 configured to map the electric activity of said stomach.

It is another object of the present invention to disclose the method as defined above, wherein said inflatable balloon 4 is comprised of an elastic polymer.

It is another object of the present invention to disclose the method as defined above, wherein said inflatable balloon 4 has size and structure configured to bring said one or more sensors 2 in contact with stomach sections such as: cardia, fundus, body and Antrum.

It is another object of the present invention to disclose the method as defined above, wherein said inflatable balloon 4 has size and structure configured to bring said one or more sensors 2 in contact with the stomach mucosa layer.

It is another object of the present invention to disclose the method as defined above, wherein said apparatus 1 capable of sensing physiological changes associated with food ingestion.

It is another object of the present invention to disclose the method as defined above, further comprising a step of filtering the stomach sensed data, such that said electrical activity is compared to data collected from a healthy stomach in order to detect abnormalities.

It is another object of the present invention to disclose the method as defined above, wherein said sensors 2 are attached to said balloon 4 is done via any known method for embedding a sensor to an expandable member.

It is another object of the present invention to disclose the method as defined above, wherein said sensors 2 have shape and size selected from the group consisting of: circular shape, elliptic shape, disc shape, and any closed curved structure, such that no piercing or any other injury or damage is preformed to said stomach wall 8.

It is another object of the present invention to disclose the method as defined above, wherein said one or more sensors 2 are combined into multi-functional sensor-platforms for neutralizing the area of said stomach wall 8 which said sensors 2 are in contact.

It is another object of the present invention to disclose the method as defined above, wherein said one or more sensors 2 are attached to said external surface 7 of said inflatable balloon 4 in a manner which prevents detaching or slipping of said sensors 2 from said balloon 4.

It is another object of the present invention to disclose the method as defined above, wherein said one or more sensors 2 are fixedly attached to said balloon 4 via thin semiconductor 13 that could bend on said balloon's external surface 7.

It is another object of the present invention to disclose the method as defined above, wherein said semiconductor 13 is sized such that they can be patterned in curves along said balloon's 4 tip.

It is another object of the present invention to disclose the method as defined above, wherein said one or more sensors 2, mounted on said semiconductor 13, are held together via springy connector 14.

It is another object of the present invention to disclose the method as defined above, further comprising a step of stimulating electrical pulses in order to provide therapy or to alleviate symptoms of obesity, bulimia or eating disorders.

It is another object of the present invention to disclose the method as defined above, wherein said inflatable balloon 4 has a mesh-like configuration 40.

It is another object of the present invention to disclose the method as defined above, wherein said mesh-like configuration 40 comprises: interconnected inflatable tubular segments 50 enclosing at least one of: polygonal-spaces and oval-spaces, and provided with an open central hollow space 53 or partially closed central hollow space 54.

It is another object of the present invention to disclose the method as defined above, wherein said polygonal-spaces are selected from a group consisting of: triangle, square 51, diamond 52, rectangular, pentagon, hexagon, heptagon, octagon and any combination thereof.

It is another object of the present invention to disclose the method as defined above, wherein said mesh-like configuration 40 is provided with at least one inflatable ring segment 55.

It is another object of the present invention to disclose the method as defined above, further comprising a step of utilizing said isopotential map for selecting a surgical solution from a list consisting of:
a. bypassing arrhythmic zones by creating a gastric bypass;
b. resecting the arrythmogenic areas by a sleeve gastrectomy or a distal gastrectomy;
c. realizing a need for total gastrectomy, in case of diffused arrhythmia not amendable for other less radical surgical options; and
d. any combination thereof.

It is another object of the present invention to disclose the method as defined above, further comprising step of providing pacing solutions, selected from the group consisting of:
a. preoperative diagnostic screening;
b. intraoperative procedures; and
c. postoperative procedures such as tuning said pacing parameters in the gastro suite, according to patient satisfaction, said electro-mechanical map and optimal energy requirements.

It is another object of the present invention to disclose the method as defined above, further comprising a step of selecting said intraoperative procedures from a group consisting of:
a. real-time localization of optimal pacing electrodes placement;
b. choosing pacing characteristics; and
c. demonstrating intraoperative electrical with said mechanical coupling map during pacing and optimization of said pacing effect.

It is another object of the present invention to disclose the method as defined above, further comprising a step of using at least one of: said catheter 20 and said sensors 2, for ablation of electrical pathways or pacemakers, for treating pathologies such as: gastroparesis, functional dyspepsia, GERD and other gastric arrhythmias, or for treating obesity by delaying gastric emptying thereby creating longer satiety time.

It is another object of the present invention to disclose the method as defined above, wherein said balloon 4 is embedded within a scaffolding-shield 60 and wherein said sensors 2 are connected to the outer surface 61 of said scaffolding-shield 60; such that said scaffolding-shield 60 immovably affixes said sensors 2 to said stomach wall 8, when said balloon 4 fully inflated.

It is another object of the present invention to disclose the method as defined above, wherein said scaffolding-shield 60 is made of elastic material selected from a group consisting of: polymers, metals, alloys, memory shapes alloys and any combination thereof.

It is another object of the present invention to disclose the method as defined above, wherein said sensors 2 are printed onto said balloon's 4 external surface 7, by three-dimensional (3D) printing techniques.

It is another object of the present invention to disclose the method as defined above, wherein said hardware-device 12 is printed onto said balloon's 4 external surface 7, adjacent to said sensors 2, by three-dimensional (3D) printing techniques.

It is another object of the present invention to disclose the method as defined above, wherein said semiconductors 13 are printed onto said balloon's 4 external surface 7, adjacent to said sensors 2, by three-dimensional (3D) printing techniques.

It is another object of the present invention to disclose an apparatus 200 configured for inserting into a mammalian stomach and adapted for sensing the activity of the stomach wall 8; said apparatus 200 comprising:
a. an expandable framework 210 insert-able into said stomach, when crimped;
b. one or more sensors 2, mounted on said framework 210;
c. a sheath 211 configured to gather said framework 210 and guide said framework 210 into and out of said stomach, when crimped; and
d. a guiding-wire 212 connected to said framework 210, configured to pull or push said framework 210 in or out of said sheath 211;
   wherein said expandable framework 210 is adapted to attach said sensors 2 to said stomach wall 8, when said framework 210 is fully expanded in said stomach; such that said sensors 2 are immovably affixed to said stomach wall 8, sufficient to sense at least one of: mechanical-activity and electric-activity, emanating from said stomach wall 8.

It is another object of the present invention to disclose the apparatus 200 as defined above, wherein said framework 210 is made of a memory shape alloy such as Nitinol.

It is another object of the present invention to disclose the apparatus 200 as defined above, wherein said framework 210 is made of a super-elastic material.

It is another object of the present invention to disclose the apparatus 200 as defined above, wherein said expandable framework 210 comprises one or more wire-like members 221, originating from the distal part of said guiding-wire 212; such that said sensors 2 are connected to the distal end of said wire-like members 221.

It is another object of the present invention to disclose the apparatus 200 as defined above, wherein said framework 210 comprises a mesh-like configuration 230; such that said sensors 2 are connected to the external surface 231 of said mesh-like configuration 230.

It is another object of the present invention to disclose the apparatus 200 as defined above, wherein said apparatus 200 is capable of measuring and detecting Gastroparesis.

It is another object of the present invention to disclose the apparatus 200 as defined above, wherein said sensors 2 are electrodes 6 selected from a group consisting of: heat, thermal or temperature electrode, mechanical electrode, electro-mechanical electrode, chemical electrode, gas electrode, electric current electrode, electric potential electrode, pressure electrode, strain electrode, acceleration electrode, piezo-electric electrode, and any combination thereof.

It is another object of the present invention to disclose the apparatus 200 as defined above, wherein said one or more sensors 2 are arranged in a non-contact multi-sensors mesh arrangement.

It is another object of the present invention to disclose the apparatus 200 as defined above, wherein said mesh arrangement is adapted for translating the stomach electrical information into a 3D or 2D isopotential map.

It is another object of the present invention to disclose the apparatus 200 as defined above, wherein said one or more sensors 2 comprises a hardware-device 12 adapted to save the data sensed by said one or more sensors 2.

It is another object of the present invention to disclose the apparatus 200 as defined above, wherein said sensors 2 are in communication with a processor 10; said communication is done by connecting said hardware-device 12 to said processor 10.

It is another object of the present invention to disclose the apparatus 200 as defined above, wherein said processor 10 is programmed to filter background electrical-noise from sensed electrical activity.

It is another object of the present invention to disclose the apparatus 200 as defined above, wherein said hardware-device 12 is mounted on either one of said one or more sensors 2.

It is another object of the present invention to disclose the apparatus 200 as defined above, wherein said communication between said processor 10 and at least one of: said sensors 2 and said hardware-device 12, are done via a communication element selected from a group comprising of: USB cable, serial cable, LAN, Bluetooth, Wi-Fi, any other element of physical or wireless connection, and any combination thereof.

It is another object of the present invention to disclose the apparatus 200 as defined above, wherein said framework 210 is mounted with a plurality of said one or more sensors 2, configured to map said electric activity of said stomach.

It is another object of the present invention to disclose the apparatus 200 as defined above, wherein said framework 210 has size and structure configured to bring said one or more sensors 2 in contact with stomach sections such as: cardia, fundus, body and Antrum.

It is another object of the present invention to disclose the apparatus 200 as defined above, wherein said inflatable said framework 210 has size and structure configured to bring said one or more sensors 2 in contact with the stomach mucosa layer.

It is another object of the present invention to disclose the apparatus 200 as defined above, wherein said apparatus 200 is capable of sensing physiological changes associated with food ingestion.

It is another object of the present invention to disclose the apparatus 200 as defined above, wherein said sensors 2 are fixedly attached to said framework 210 via any known method for embedding a sensor to an expandable member.

It is another object of the present invention to disclose the apparatus 200 as defined above, wherein said sensors 2 are fixedly attached to said framework 210 via thin semiconductors 13 bend-able on said framework 210.

It is another object of the present invention to disclose the apparatus 200 as defined above, wherein said semiconductors 13 are sized such that they can be attached to said framework 210.

It is another object of the present invention to disclose the apparatus 200 as defined above, wherein said one or more sensors 2 mounted on said semiconductor 13 are held together via springy connectors 14.

It is another object of the present invention to disclose the apparatus 200 as defined above, wherein said sensors 2 have shape and size selected from the group consisting of: circular shape, elliptic shape, disc shape, and any closed curved structure, such that no piercing or any other injury or damage is preformed to said stomach wall 8.

It is another object of the present invention to disclose the apparatus 200 as defined above, wherein said sensors 2 are combined into multi-functional sensor-platforms for neutralizing the area of said stomach wall 8 which said sensors 2 are in contact with.

It is another object of the present invention to disclose the apparatus 200 as defined above, wherein said sensors 2 are attached to said framework 210, in a manner which prevents detaching or slipping of said sensors 2 from said framework 210.

It is another object of the present invention to disclose the apparatus 200 as defined above, wherein said one or more electrodes 6 are adapted for stimulating electrical-pulses in order to provide therapy or to alleviate symptoms of obesity, bulimia or eating disorders.

It is another object of the present invention to disclose a method for detecting stomach activity, said method comprising steps of:
a. providing an expandable apparatus 200 comprising:
   i. an expandable framework 210 insert-able into said stomach, when crimped;
   ii. one or more sensors 2, mounted on said framework 210;
   iii. a sheath 211 configured to gather said framework 210 and guide said framework 210 into and out of said stomach, when crimped; and
   iv. a guiding-wire 212 connected to said framework 210, configured to pull or push said framework 210 in or out of said sheath 211;
b. inserting said apparatus 200 comprising said framework 210 in its crimped configuration into a patient hollow stomach system;
c. orienting said framework 210 adjacent to stomach wall 8, thereby contacting said sensors 2 with said stomach wall 8;
d. pushing said framework 210 out of said sheath 211 to an immovably affixed contact with the stomach wall 8;
e. sensing at least one of: electrical-activity and mechanical-activity, by said one or more sensors 2;
f. communicating said sensed electrical activity from said one or more sensors 2 to a processor 10; and
g. filtering background electrical noise from the sensed electrical activity;
wherein said framework 210 is adapted for attaching said one or more sensors 2 to said stomach wall 8, when framework 210 is fully expanded in said stomach; thereby said sensors 2 are immovably affixed to said stomach wall 8, sufficient for sensing at least one of: mechanical-activity and electric-activity, emanating from said stomach wall 8.

It is another object of the present invention to disclose the method as defined above, wherein said framework 210 is made of a memory shape alloy such as Nitinol.

It is another object of the present invention to disclose the method as defined above, wherein said framework 210 is made of a super-elastic material.

It is another object of the present invention to disclose the method as defined above, wherein said expandable framework 210 comprises one or more wire-like members 221 originating from the distal part of said guiding-wire 212; such that said sensors 2 are connected to the distal end of said wire-like members 221.

It is another object of the present invention to disclose the method as defined above, wherein said framework 210 comprises a mesh-like configuration 230; such that said sensors 2 are connected to the external surface 231 of said mesh-like configuration 230.

It is another object of the present invention to disclose the method as defined above, wherein said apparatus 200 is capable of measuring and detecting Gastroparesis.

It is another object of the present invention to disclose the method as defined above, wherein said sensors 2 are electrodes 6 selected from a group consisting of: heat, thermal or temperature electrode, mechanical electrode, electro-mechanical electrode, chemical electrode, gas electrode, electric current electrode, electric potential electrode, pressure electrode, strain electrode, acceleration electrode, piezo-electric electrode, and any combination thereof.

It is another object of the present invention to disclose the method as defined above, wherein said one or more sensors 2 are arranged in a non-contact multi-sensors mesh arrangement.

It is another object of the present invention to disclose the method as defined above, wherein said mesh arrangement is adapted for translating the stomach electrical information into a 3D or 2D isopotential map.

It is another object of the present invention to disclose the method as defined above, wherein said one or more sensors 2 comprises a hardware-device 12 adapted to save the data sensed by said one or more sensors 2.

It is another object of the present invention to disclose the method as defined above, wherein said sensors 2 are in communication with a processor 10; said communication is done by connecting said hardware-device 12 to said processor 10.

It is another object of the present invention to disclose the method as defined above, wherein said processor 10 is programmed to filter background electrical-noise from sensed electrical activity.

It is another object of the present invention to disclose the method as defined above, wherein said hardware-device 12 is mounted on either one of said one or more sensors 2.

It is another object of the present invention to disclose the method as defined above, wherein said communication between said processor 10 and at least one of: said sensors 2 and said hardware-device 12, are done via a communication element selected from a group comprising of: USB cable, serial cable, LAN, Bluetooth, Wi-Fi, any other element of physical or wireless connection, and any combination thereof.

It is another object of the present invention to disclose the method as defined above, wherein said framework 210 is mounted with a plurality of said one or more sensors 2, configured to map said electric activity of said stomach.

It is another object of the present invention to disclose the method as defined above, wherein said framework 210 has size and structure configured to bring said one or more sensors 2 in contact with stomach sections such as: cardia, fundus, body and Antrum.

It is another object of the present invention to disclose the method as defined above, wherein said inflatable said framework 210 has size and structure configured to bring said one or more sensors 2 in contact with the stomach mucosa layer.

It is another object of the present invention to disclose the method as defined above, wherein said apparatus 200 is capable of sensing physiological changes associated with food ingestion.

It is another object of the present invention to disclose the method as defined above, wherein said sensors 2 are fixedly attached to said framework 210 via any known method for embedding a sensor to an expandable member.

It is another object of the present invention to disclose the method as defined above, wherein said sensors 2 are fixedly attached to said framework 210 via thin semiconductors 13 bend-able on said framework 210.

It is another object of the present invention to disclose the method as defined above, wherein said semiconductors 13 are sized such that they can be attached to said framework 210.

It is another object of the present invention to disclose the method as defined above, wherein said one or more sensors 2 mounted on said semiconductor 13 are held together via springy connectors 14.

It is another object of the present invention to disclose the method as defined above, wherein said sensors 2 have shape and size selected from the group consisting of: circular shape, elliptic shape, disc shape, and any closed curved structure, such that no piercing or any other injury or damage is preformed to said stomach wall 8.

It is another object of the present invention to disclose the method as defined above, wherein said sensors 2 are combined into multi-functional sensor-platforms for neutralizing the area of said stomach wall 8 which said sensors 2 are in contact with.

It is another object of the present invention to disclose the method as defined above, wherein said sensors 2 are attached to said framework 210, in a manner which prevents detaching or slipping of said sensors 2 from said framework 210.

It is still an object of the present invention to disclose the method as defined above, wherein said one or more electrodes 6 are adapted for stimulating electrical-pulses in order to provide therapy or to alleviate symptoms of obesity, bulimia or eating disorders.

It is lastly an object of the present invention to disclose the method as defined above, further comprising step of pulling said framework 210 into the sheath 211 and pulling said apparatus 200 out of said stomach.

### BRIEF DESCRIPTION OF THE FIGURES

In order to understand the invention and to see how it may be implemented in practice, a few preferred embodiments will now be described, by way of non-limiting example only, with reference to be accompanying drawings, in which:

FIGS. 1A and 1B are side views of an expandable apparatus for detecting stomach electrical activity, in accordance with a preferred embodiment of the present invention;

FIG. 1C is a magnified view of the sensing sensor and its accompanying components;

FIG. 2 is a view of the sensing system for detecting electrical signals of the stomach movement, in accordance with a preferred embodiment of the present invention;

FIG. 3 is a schematic view illustrating the apparatus according to an embodiment of the invention being introduced into the stomach of a patient for measuring and detecting electro-mechanical signals, in accordance with a preferred embodiment of the present invention;

FIG. 4 is a schematic diagram illustrating steps of the inventive method, in accordance with a preferred embodiment of the present invention;

FIGS. 5A, 5B, 6A, 6B, 7A, 7B, 8A, 8B and 8C are different views of several configurations for the inflatable balloon mesh-like configuration;

FIG. 9 is a schematic view illustrating the apparatus according to an embodiment of the invention where the balloon is within a scaffolding shield which carries the sensors;

FIGS. 10A and 10B are schematic views of expandable apparatus comprising a framework with wire-like members, according to an embodiment of the invention, shown in expanded (FIG. 10A) and (FIG. 10B) crimped positions; and

FIGS. 11A and 11B are schematic views of expandable apparatus comprising a framework with mesh-like configuration, according to an embodiment of the invention, shown in expanded (FIG. 11A) and (FIG. 11B) crimped positions.

### DETAILED DESCRIPTION OF THE INVENTION

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide device, method and system for diagnosing Gastroparesis by detecting electro-mechanical signals.

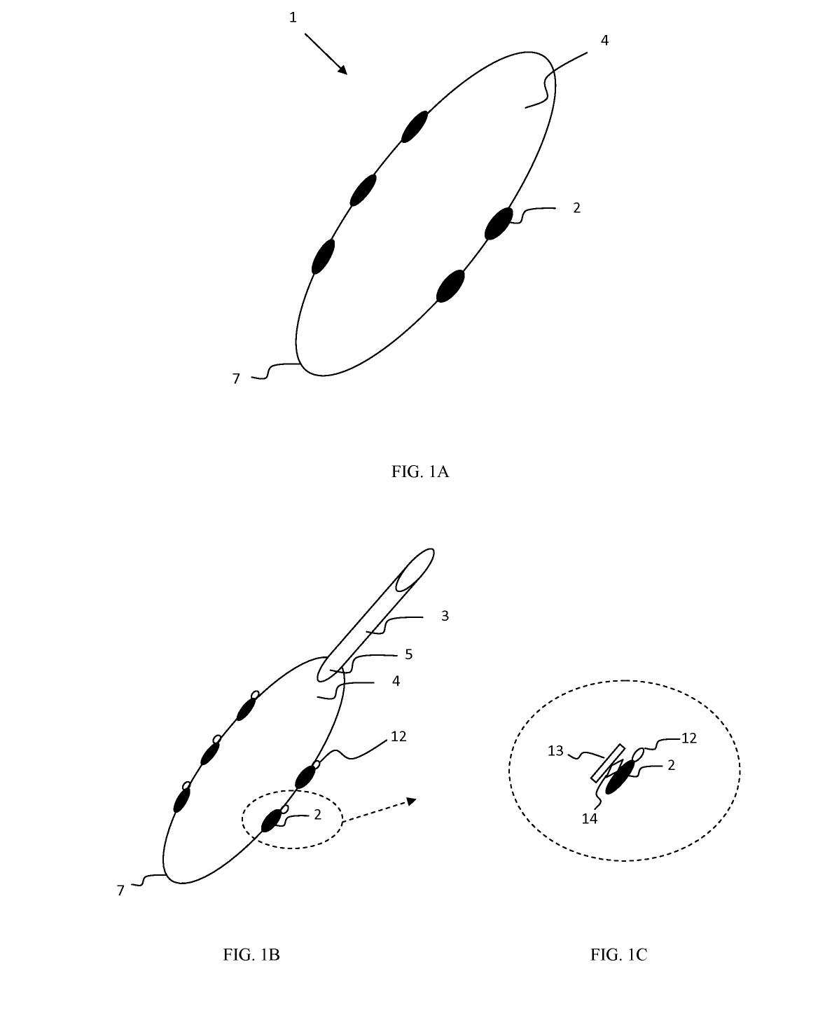

The present invention embodied herein relates to an apparatus, system and method for mapping electrical activity in the stomach in order to diagnose and also treat gastric motility disorders. The present invention provides means for detecting electro-mechanical coupling correlating to the movement and motility of the stomach or other hollow organs. Another aspect of the invention is to provide therapeutic means and methods for stimulating the stomach into improved movement. Embodiments of the invention include in FIG. 1B a tube 3 such as a catheter 20 with an inflatable balloon 4 coupled with electrodes 6 and/or sensors 2 into the stomach. The balloon 4 is designed to be inflated until the balloon 4 abuts the inner wall of the stomach in order for the balloon 4 and the catheter 20 to be fixed in relation to the stomach. The external surface 7 of the balloon 4 carries several sensors 2 and/or electrodes 6 which are brought into contact with the stomach wall 8, when the balloon 4 is fully inflated in the stomach.

The stomach is comprised of several layers. The inner layer is the mucosa. The next layer is the submucosa followed by the outer muscular layers. Surrounding the muscular layers is the serosal layer.

Reference to “stomach wall” or “wall of the stomach” as used herein include the entire thickness of the stomach, including the mucosa, submucosa, muscular layers, and serosa. The “anterior wall of the stomach” is the portion of the stomach closest to the muscular abdominal wall and the “posterior wall of the stomach” is the part of the stomach closest to the retroperitoneum. It is herein acknowledged that many of the embodiments of the invention herein described will be applied to other hollow body organs with minor conventional alterations within the abilities of a person skilled in the art.

The term “measuring means” as used herein, include sensors and electrodes.

The term “sensor”, as used herein, is a converter that measures a physical quantity and converts it into a signal, which can be read by an observer or by an instrument.

The term “electrode”, as used herein, is an electrical conductor used to make contact with a nonmetallic part of a circuit (e.g. a semiconductor, an electrolyte or a vacuum).

The term “attach” as used herein, means cause to attach, connect, link, or abut.

The term “shape-memory alloy” as used herein, is an alloy that remembers its original shape. Nickel titanium, also known as Nitinol, is a metal alloy of nickel and titanium, where the two elements are present in roughly equal atomic percentages. Nitinol alloys exhibit two closely related and unique properties: shape memory and super-elasticity (also called pseudo-elasticity).

The term “framework” as used herein, is a structure for supporting or containing something.

The term “3D printing” as used herein, is a process of additive manufacturing for making a three-dimensional (3D) solid object.

Reference is now made to FIG. 1A which illustrates the expandable apparatus 1 for reinforcing one or more sensors 2 against the stomach surface. The apparatus comprises: (a) an inflated balloon 4 adapted to be inserted into inner cavity of a mammalian stomach, and (b) one or more sensors 2 mounted to the external surface 7 of the balloon 4. The balloon 4 is designed to be inflated until the balloon 4 abuts the inner wall of the stomach. The inflatable balloon 4 is adapted to bring the one or more sensors 2 into substantially immobile contact with the stomach wall 8 when the balloon 4 is fully inflated in the stomach; the sensors 2 are immovably affixed to the stomach wall 8 sufficient to sense an electric- and/or mechanical-activity emanating from the stomach wall 8.

FIG. 1B further illustrates a sensing apparatus for detecting stomach electrical- and/or mechanical-activity, the apparatus comprises: (a) an inflatable balloon 4 adapted to be inserted into a mammalian stomach, (b) one or more sensors 2 which are mounted to the external surface 7 of the balloon 4, and (c) a tube 3 connected on one end 5 to the balloon 4. The tube 3 is adapted for inflating and/or deflating the balloon 4.

The balloon 4 is designed to be inflated until the balloon 4 abuts the inner wall of the stomach, in order for the balloon 4 and the tube 3 to be fixed in relation to the stomach. The external surface 7 of the balloon 4 carries one or more sensors 2 in a manner which reinforces the sensors 2 to the balloon's external surface 7, when the balloon 4 is fully inflated in the stomach. The sensors 2 configured for secure fixation to the stomach wall 8, when the balloon 4 is fully inflated in the stomach. The sensors 2 abut the inner wall 8 of the stomach in an immobile manner sufficient to sense an electric activity emanating from the stomach wall 8.

Reference is now made to FIG. 1C. The manner for attaching the sensors 2 to the balloon 4 can be performed via a variety of methods. One of these methods may include thin semiconductors 13 that could bend on the surface. The electrodes 6 and/or sensors 2 are mounted on semiconductors 13, which can be patterned in curves along the balloon's 4 tip. Springy connectors 14 hold the sensors 2 together such that the electronics function normally while the balloon 4 is inflated or deflated.

In another embodiment the sensors 2 and/or electrodes 6 are printed onto the balloon's 4 external surface 7, by three-dimensional (3D) printing techniques. In another embodiment the at least one hardware-device 12 and/or the semiconductors 13 are printed onto the balloon's 4 external surface 7 adjacent to said sensors 2 and/or electrodes 6, by three-dimensional (3D) printing techniques.

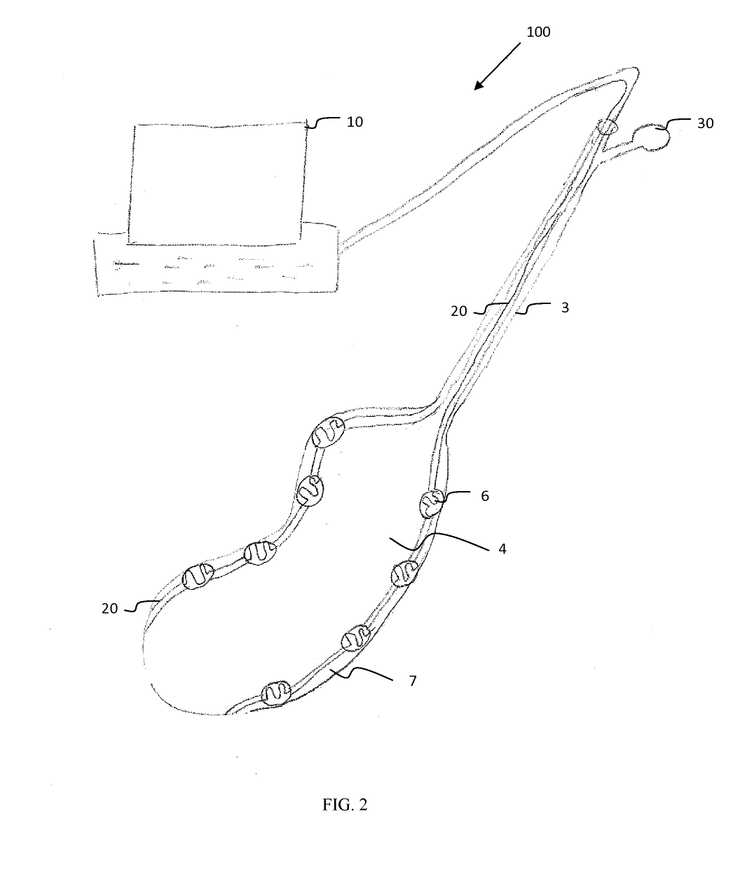

Reference is now made to FIG. 2 which illustrates a system 100 for detecting stomach electrical activity, the system 100 comprises (a) an inflatable intragastric balloon 4 adapted to be inserted into a patient stomach, (b) one or more electrodes 6, which are mounted to the external surface of the balloon 4, (c) a tube 3, connected on one end 5 to the balloon 4, configured to inflate and deflate the balloon 4 and (d) a processor 10 in communication with the electrodes 6.

The balloon 4 is made from a material readily extendable such that it is non-harmful to the human or animal body. The balloon 4 has distinct physical properties such as elasticity modus of strain etc. The balloon 4 is made from an elastic polymer. The thickness of the material, which the balloon 4 is made of, is between about 0.2 mm and about 0.5 mm.

However, any other well-suited material may be used as long as it fulfils the need for expandability, security and biocompatibility. The balloon 4 is adapted to be inflated and, or deflated. The inflating and or deflating process are achieved through connectors or via any suitable tube such as catheter 20 known in the art. The catheter 20 further includes a valve 30 for activating the catheter 20 and the balloon's 4 inflating process. The inflation process is preformed in order to bring the electrodes 6 in a physical contact with the stomach wall 8.

The balloon 4 is introduced into the stomach through the esophagus using the trans-esophageal delivery system such as a tube 3, a catheter 20 or a sheath as illustrates in FIG. 2. For example, the balloon 4, in its un-inflated state, is contained within a tube 3, catheter 20 or sheath which acts as an introducer. When the balloon 4 is placed within the stomach a biocompatible fluid or a gas may be infused into the inflatable means such that its inflated body expands to fill in most of the lumen of the stomach including the fundus and pyloric part. A valve 30 means may be provided for sealing it off and may permit further evacuating the fluid or gas off the body, such that the balloon 4 can be brought back into the non-expanded state providing for its removal off the stomach through the esophagus. The device may be inflated with fluid such as but not limited to saline, by gas or by gels. The geometrical shape of the apparatus when is expanded prevents it from being displaced from the stomach through the pylorus or back into the esophagus.

The tube 3 may also be a detachable tube. In such case, the distal end is provided with a cannula, which is inserted through a septum attached to the inflation port of the apparatus. In order to release the tube from the inflation port, the tube should be pulled with a predetermined force. Inflation of the balloon 4 should be controlled by means of a suitable pressure gauge as known.

The sensors 2 are adapted to sense electric- and/or mechanical-activity when it is in contact with the stomach mucosa. The sensors 2 and/or electrodes 6 comprise a hardware-device 12 adapted to save data sensed by the one or more sensors 2 or electrodes 6.

Each of the sensor 2 can be an electrode 6 selected from a group consisting of: heat, thermal or temperature electrode, mechanical electrode, electro-mechanical electrode, chemical electrode, gas electrode, electric current electrode, electric potential electrode, pressure electrode, acceleration electrode, and any combination thereof.

The inflatable balloon 4 can be mounted with a plurality of sensors 2 and/or electrodes 6 located lengthwise the balloon 4 in order to map the electric- and or mechanical-activity of large portions of the stomach surface. The sensors 2 and/or electrodes 6 attached directly onto the balloon 4 such that the sensors 2 and/or electrodes 6 can provide accurate feedback of the desired information. The sensors 2 and/or electrodes 6 are attached to the balloon 4 by the manner selected from a group consisting of: gluing, welding, grafting, assembling, mounting, or any known method suitable for embedding an electric electrode on an expandable member, guaranteeing a reliable fixation of the sensors 2 and/or electrodes 6 to the balloon's external surface 7.

In another embodiment of the invention, several types of electrodes 2 which are designed to be attached on a surface and also can be used as an implant on the balloon 4 surface, for example: spiral electrodes, cuff electrodes, steroid eluting electrodes, wrap-around electrodes, or hydrogel electrodes. The local electrodes or mechanical sensors may comprise stitch, coil, screw, patch, basket, needle and/or wire electrodes, or substantially any other electrode known in the art of electrical stimulation or sensing within the body. The sensors 2 and/or electrodes 6 are made of a flexible material that gives less stimulation to the mucosa.

The apparatus may further have a variety of detecting means inserted into the balloon 4, the detecting means preferably being chosen among means such as: strain measuring means, pressure measuring means, temperature measuring means, piezo-electrical measuring means, ultrasonic measuring means or means for recording flow of fluid. The apparatus can also measure a number of artificially applied stimuli, such as: mechanical stimulus, thermal stimulus, chemical stimulus and electric stimulus.

In another embodiment of the invention, piezoelectric sensors can be used in order to measure pressure, acceleration, strain or force of the stomach wall 8 and to convert them to an electrical charge by using piezoelectric effect. An array of piezo-electric elements may be arranged in the balloon member. The array of piezo-electric elements can be used for ultrasonic scanning of body cavities and also capable of scanning the pain inflicted on the patient during insertion of a probe into a body cavity. The piezoelectric transducer has very high DC output impedance and can be modeled as a proportional voltage source and filter network. The voltage at the source is directly proportional to the applied force, pressure, or strain. The output signal is then related to this mechanical force as if it had passed through the equivalent circuit.

The activity sensed by the sensors 2 and/or electrodes 6 mounted to the inflatable balloon 4 is transmitted to a processor 10 which is programmed to filter background electrical noise from the sensed electrical activity.

The apparatus may further comprise non-contact multi-electrodes which together constitute a mesh arrangement. This mesh arrangement can collect rapidly real-time stomach electrical information and translate it into a three-dimensional (3D) or 2D isopotential map. The non-contact multi-electrodes structure is based on an electrophysiology procedure and configuration having the ability to collect more than 3,000 points of electrical data. It will allow physicians to appropriately locate and diagnose stomach movement and interruption.

The communication between the sensors 2 and/or electrodes 6 and the processor 10 is done by connecting hardware-device 12 to the processor 10. The hardware-device 12 is mounted on either one of the electrical electrode 6, meaning to the external surface 7 of the balloon 4 or the internal surface. The communication between hardware-device 12 and the processor 10 is done via a communication method for delivering electric signals, selected from a group comprising of USB cable, serial cable, LAN, Bluetooth, Wi-Fi, any other method of physical or wireless connection which is known in the art. The data detected by the electrodes 2 is accumulated and analyzed in order to filter the data corresponding to stomach movement from any noise (i.e. movement of any other internal organs or external noise detected by the electrodes 2), the filtered data is then compared to data collected from a healthy stomach in order to detect abnormalities. By transforming the mechanical action into an electrical impulse the system 100 describes the electromechanical coupling function of the stomach.

Filtering is a class of signal processing which removes from a signal some unwanted component or feature. The defining feature of filters is the complete or partial suppression of some aspect of the signal. Most often, this means removing some frequencies and not others in order to suppress interfering signals and reduce background noise. There are many different bases of classifying filters and these overlap in many different ways; there is no simple hierarchical classification. Filters may be: analog or digital, discrete-time (sampled) or continuous-time, linear or non-linear, time-invariant or time-variant, also known as shift invariance. If the filter operates in a spatial domain then the characterization is space invariance, passive or active type of continuous-time filter, infinite impulse response (IIR) or finite impulse response (FIR) type of discrete-time or digital filter. Filters can be built in a number of different technologies. The same transfer function can be realized in several different ways, that is the mathematical properties of the filter are the same but the physical properties are quite different. Often the components in different technologies are directly analogous to each other and fulfill the same role in their respective filters. For instance, the resistors, inductors and capacitors of electronics correspond respectively to dampers, masses and springs in mechanics. Likewise, there are corresponding components in distributed element filters. Electronic filters were originally entirely passive consisting of: resistance, inductance and capacitance. Active technology makes design easier and opens up new possibilities in filter specifications.

Digital filters operate on signals represented in digital form. The essence of a digital filter is that it directly implements a mathematical algorithm, corresponding to the desired filter transfer function, in its programming or microcode.

Mechanical filters are built out of mechanical components. In the vast majority of cases they are used to process an electronic signal and transducers are provided to convert this to and from a mechanical vibration. However, examples do exist of filters that have been designed for operation entirely in the mechanical domain.

Distributed element filters are constructed out of components made from small pieces of transmission line or other distributed elements. There are structures in distributed element filters that directly correspond to the lumped elements of electronic filters, and others that are unique to this class of technology.

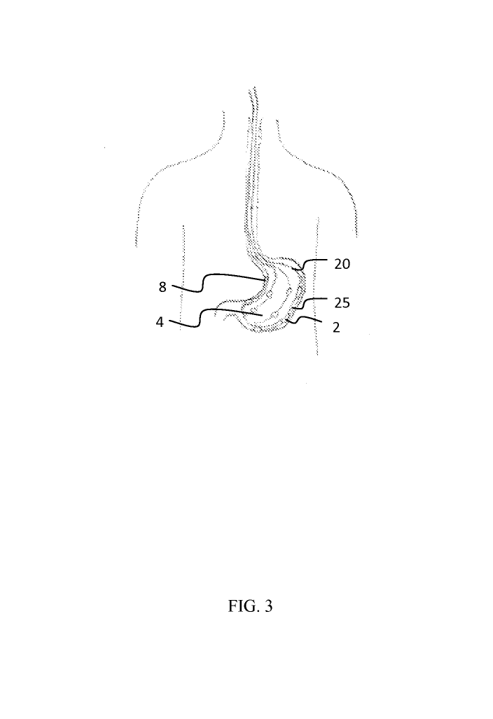

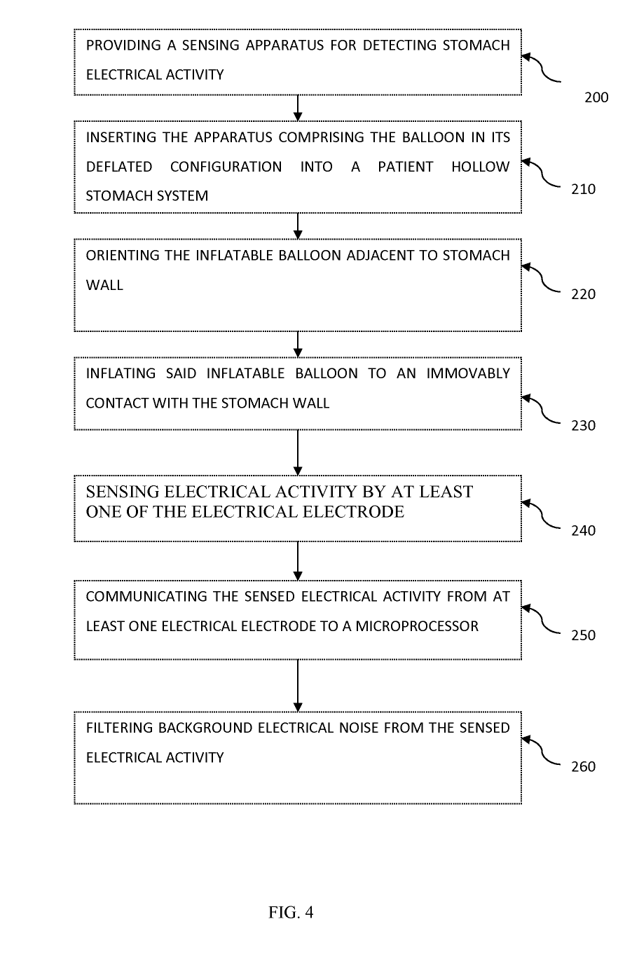

Reference is now made to FIG. 3 which illustrates the apparatus 1 of the present invention being introduced into the stomach of a patient for measuring and detecting electro-mechanical signals. The balloon 4 is an expandable member which has size and structure such that when it is placed within the interior of the stomach the electrodes 6 are in contact with the stomach mucosa.

The inflatable balloon 4 has size and structure to attach the one or more sensors 2 and/or electrodes 6 in contact with stomach section such as: cardia, fundus, body and Antrum. The sensors 2 and/or electrodes 6 in a form of contact electrodes 2 are attached to the balloon 4 via any known acceptable mechanism for embedding an electrode to an expandable member. The electrodes are fixedly connected to the external surface of the inflatable balloon 4 in a secured manner which prevents slipping or detaching of the sensors 2 and/or electrodes 6 from the balloon's external surface 7. When the balloon 4 is introduced to the stomach via the esophagus, the inflatable balloon 4 comprising the mounted sensors 2 and/or electrodes 6 designed and acts as one integrated unit and inserted without any interruption. The balloon 4 and the catheter 20 are fixed in relation to the esophagus and the stomach, resulting in a reliable fixation of the sensors 2 and/or electrodes 6 to the stomach wall 8. The method and wiring of the sensors 2 and/or electrodes 6 to the processor 10 may carried out in any convenient manner as known by a person skilled in the art.

In order to prevent injury of the stomach wall such as perforation, scratching, bleeding, irritation, or tissue-piercing or any other damage, the sensors 2 and/or electrodes 6 are shaped and have a geometric structure selected from the group consisting of: helix shaped, spiral cuff shape, circular shape, elliptic shape, disc shape, or any curved and flexible structure known in the art for electric electrode which has the properties and configuration to be attached and adjacent to an expandable member.

The system 100 is further capable of sensing physiological changes associated with food ingestion.

In another embodiment of the present invention, the procedure of inserting the expandable apparatus 1 can be performed via a catheter 20 as is done in the heart, using RF (Radio Frequency) to foci of interest (pacemakers, arrythmogenic foci). This procedure involves inserting a catheter 20, a small flexible tube through the esophagus and following the esophagus's path into the stomach. Once the tube 3 is in place, the balloon 4 is inflated within the gastric lumen, in touch with the mucosal layer and signals from the sensors 2 and/or electrodes 6 are received and processed in order to create an electrical and mechanical map of the stomach.

In addition, catheter ablation can be preformed following the mapping of the gastric electromechanical activity by the expandable apparatus 100. In this procedure, the ablation catheter is navigated to contact with the tissue through the balloon's 4 openings, or hollow spaces 53, 54.

In accordance with the preferred embodiment of the present invention, the electrodes 2 are combined into multi-functional electrode platforms for neutralizing the area of the stomach wall 8, where the sensors 2 and/or electrodes 6 are in contact. The insertion and retraction procedure can be performed in an endoscopic manner such that the apparatus is inserted directly into the organ for examine the interior of a hollow organ or cavity of the body.

The system may further provide a number of canals 25 which are running in the inside and/or the outside the surface of the catheter 20 and the attached balloon 4. Some of the canals 25 may be intended for passing, stimulating, ablating or measuring means from the balloon 4 located in the proximal end of the catheter 20 to a more distant end of the catheter 20. Those canals 25 may be provided for passing electrical wires for performing and delivering electric signals, the canals 25 may be provided also for passing a chemical substance for performing chemical stimuli, or the canals 25 may be provided for passing electrical wires and other recording means attached to the balloon 4, provided inside of the balloon 4, or attached or provided elsewhere along the extension of the catheter 20.

The invention further relates to the use of an apparatus for performing the method according to the first aspect for stimulating a part of the digestive system including the stomach and the bowel or for stimulating a part of the urogenital system including the urinary bladder, or for stimulating part of the cardiovascular system including the heart, or for stimulating part of the reproductive system including the uterus by any of the following stimuli: mechanical stimulus, thermal stimulus, chemical stimulus and electric stimulus

The electrical wires may be provided in connection with means for measuring electric signals and, or means for recording parameters such as temperature, local pressure, area force or any other physical property in relation to using the apparatus for measuring. For performing measurements of a force applied by the wall 8 of stomach strains may be attached to the catheter 20. The pressure can be measured by pressure sensors 2 and/or electrodes 6 and monitored, possibly by a pressure gauge.

Further utilizations of the apparatus, system and method can be for detecting cancer or treating obesity or weight disorders in a subject. The apparatus may be capable of sensing physiological changes associated with food or hunger and a mechanism adapted for directly stimulating or ablating a region responsive to a gastrointestinal satiety agent or for manipulating gastric emptying time.

In accordance with the preferred embodiment of the present invention, a method for detecting stomach electrical activity, the method comprising steps of:

(a) providing 200 a sensing apparatus for detecting stomach electrical activity, the apparatus comprising: (i) an inflatable balloon 4 adapted to be inserted into a mammalian stomach, (ii) one or more sensors 2, the sensors 2 are mounted on external surface 7 of the balloon 4, and (iii) a tube 3 connected on one end 5 to the balloon 4. The tube 3 is adapted for inflating and or deflating the balloon 4.

(b) inserting 210 the apparatus comprising the balloon 4 in its deflated configuration into a patient hollow stomach system,

(c) orienting 220 the inflatable balloon 4 adjacent to stomach wall 8 such that the electrical electrodes 2 are in contact with the stomach wall 8,

(d) inflating 230 the inflatable balloon 4 to an immovably affixed contact with the stomach wall 8,

(e) sensing 240 electrical activity by at least one of the electrical electrode 2,

(f) communicating 250 the sensed electrical activity from at least one electrical electrode 2 to a processor, and

(g) programming 260 the processor to filter background electrical noise from the sensed electrical activity.

The external surface 7 of the balloon 4 carries one or more sensors 2 in a manner which reinforces the sensors 2 to the balloon's external surface 7, when the balloon 4 is fully inflated in the stomach.

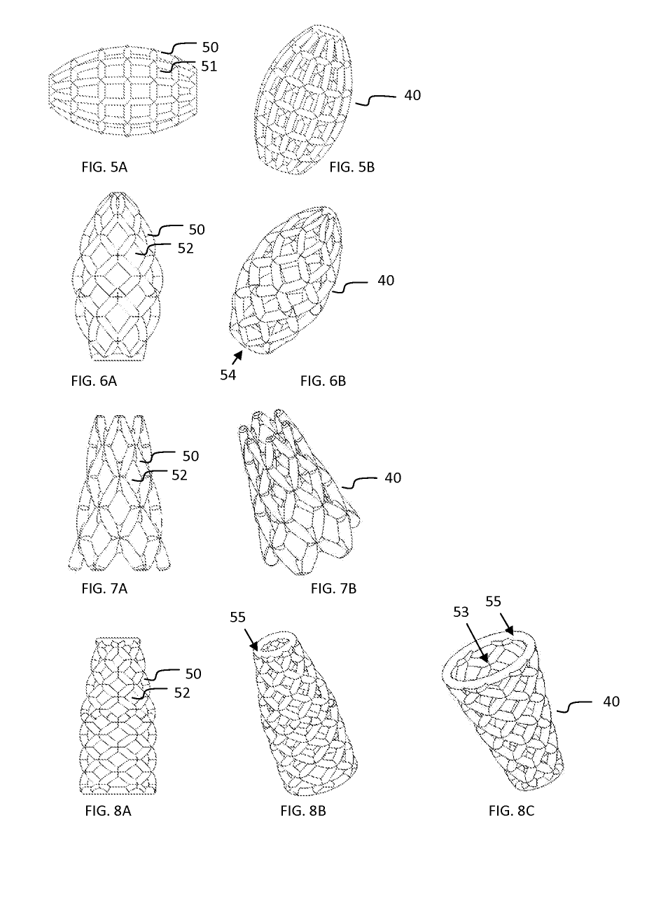

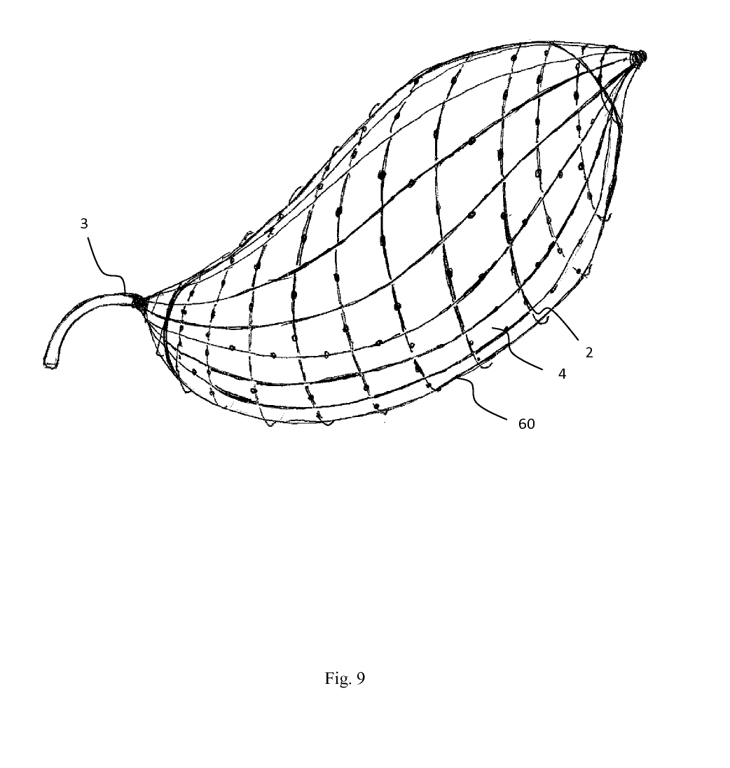

The inflatable balloon 4, according to another embodiment of the present invention, has a mesh-like configuration 40, as shown in FIGS. 5A, 5B, 6A, 6B, 7A, 7B, 8A, 8B and 8C demonstrating different views of the different configurations. The size of the mesh-like configuration 40 corresponds to the size of the stomach wall 8 in which it is to be implanted. The mesh-like configuration 40 is created by interconnected inflatable tubular segments 50 enclosing square-spaces 51, as shown in FIGS. 5A and 5B, or diamond-spaces 52, as shown in FIGS. 6A, 6B, 7A, 7B, 8A, 8B and 8C. The mesh-like configuration 40 is therefore provided with an open-central hollow space 53, as shown in FIGS. 7A, 7B, 8A, 8B and 8C, or partially closed-central hollow space 54, as shown in FIGS. 6A and 6B. The mesh-like configuration 40 may be further provided with at least one inflatable ring segment 55, as shown in FIGS. 8A 8B and 8C. This inflated mesh-like configuration 40 provides for a predictable force and pressure to be exercised on the stomach wall 8.

Reference is now made to FIG. 9 which is a schematic view illustrating the apparatus 1 according to another embodiment of the invention, where the balloon 4 is embedded within a scaffolding-shield 60, which carries the sensors 2 towards the stomach-wall 8. When the balloon 4 is inflated within the scaffolding-shield 60 it guides the scaffolding-shield 60 towards the mucosal surface or the stomach wall 8 and compels the geometry of the scaffolding-shield 60 to fit into the stomach cavity, thereby the scaffolding shield 60 immovably affixes the sensors 2 to the stomach wall 8. The scaffolding-shield 60 can be made of a material selected from a group consisting of: polymers, metals, alloys, memory shapes alloys and any combination thereof.

In another embodiment, the present invention discloses an expandable apparatus 200 configured for inserting into a mammalian stomach and adapted for sensing the activity of the stomach wall 8. The apparatus 200 comprising:
a. an expandable framework 210 insert-able into the stomach, when crimped;
b. one or more sensors 2, mounted on the framework 210;
c. a sheath 211 configured to gather the framework 210 and guide the framework 210 into and out of the stomach, when crimped; and
d. a guiding-wire 212 configured to pull or push the framework 210 in or out of the sheath 211;

The expandable framework 210 is adapted to attach the one or more sensors 2 to the stomach wall 8, when the framework 210 is out of said sheath and fully expanded in the stomach; such that the sensors 2 are immovably affixed to the stomach wall 8, sufficient to sense mechanical- and/or electric-activity emanating from the stomach wall 8.

The framework 210 is made of a shape-memory alloy that remembers its original expanded shape, such as Nickel titanium, also known as Nitinol. Nitinol is a metal alloy of nickel and titanium, where the two elements are present in roughly equal atomic percentages. Nitinol alloys exhibit two closely related and unique properties: shape memory and super-elasticity (also called pseudo-elasticity).

The shape of the expandable framework 210 can be formed of one or more wire-like members 221 originating the distal part of the guiding-wire 212. The sensors 2 are connected to the distal end of the wire-like members 221.

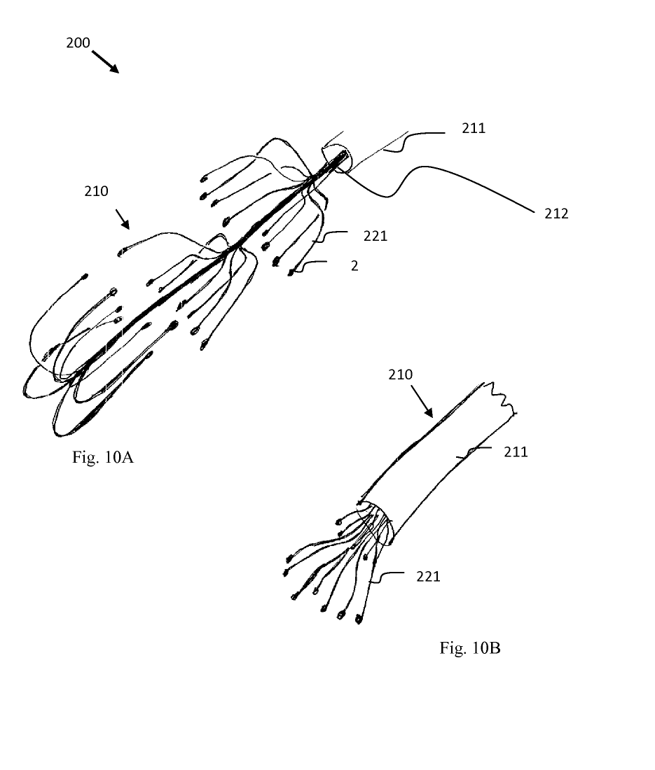

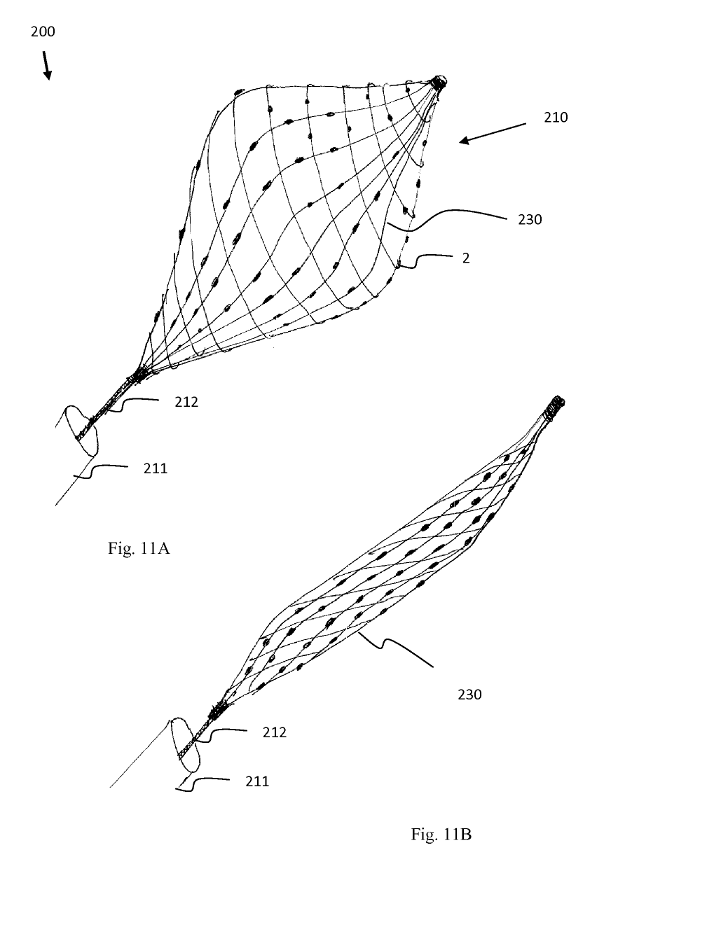

FIGS. 10A and 10B are schematic views of expandable apparatus 200 comprising a framework 210 with wire-like members 221, shown in expanded (FIG. 10A) and (FIG. 10B) crimped positions.

In yet another embodiment the expandable framework 210 can be formed of a mesh-like configuration 230, where the sensors 2 are connected to the external surface 231 of the mesh-like configuration 230.

FIGS. 11A and 11B are schematic views of expandable apparatus 200 comprising a framework 210 with mesh-like configuration 230, shown in expanded (FIG. 11A) and (FIG. 11B) crimped positions, it is shown that the mesh-like configuration is connected to the guiding rod 212 configured to push or pull the mesh-like configuration out-of or in-to the sheath 211.

The present invention discloses the following treatment methods:
utilizing the electrical map of the stomach for possible surgical solutions for dysmotility selected from the group consisting of:
   a. bypassing arrhythmic zones by creating a gastric bypass;
   b. resecting the arrythmogenic areas by a sleeve gastrectomy or a distal gastrectomy;
   c. realizing the need for total gastrectomy, in case of diffused arrhythmia not amendable for other less radical surgical options; and
   d. any combination thereof;
providing pacing solutions, selected from the group consisting of:
   a. preoperative diagnostic screening; thereby finding the patients that are most likely to benefit from the pacing, type of pacing (e.g. high amplitude—slow frequency, low amplitude—high frequency, sequential, etc.)
   b. intraoperative procedures, such as:
      i. real-time localization of optimal pacing electrodes placement;
      ii. choosing pacing characteristics; and
      iii. demonstrating intraoperative electrical with a mechanical coupling map during pacing and optimization of the pacing effect;
   c. postoperative procedures, such as tuning of pacing parameters in the gastro suite according to patient satisfaction, electro-mechanical map and optimal energy requirements;
using the catheter 20 and/or the electrode 6 for ablation of electrical pathways or pacemakers (laser, radiofrequency, electrocautery), for treating pathologies such as: gastroparesis, functional dyspepsia, GERD and other gastric arrhythmias, or for treating obesity by delaying gastric emptying thereby creating longer satiety time.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1-161. (canceled)

162. A system for detecting electrical activity of the stomach, the system comprising:
an inflatable balloon configured to be inserted, when deflated, into the stomach;
multiple electrodes mounted on the external surface of said balloon and arranged as a mesh;
a tube connected to a proximal end of said balloon and configured to inflate and deflate said balloon; and
a processor in communication with said one or more electrodes,
wherein:
said balloon is configured, when inflated, to bring said multiple electrodes into substantially immobile contact with the stomach wall,
said electrodes are configured to sense electrical activity emanating from said stomach wall and to transmit a signal corresponding to the activity to said processor, and
said processor is programmed to translate the signal into an electrical activity map of the stomach.

163. The system according to claim 162, wherein said electrical activity map is selected from the group consisting of: a three-dimensional isopotential map and a two-dimensional isopotential map.

164. The system according to claim 162, wherein said multiple electrodes are physically connected to said processor.

165. The system according to claim 162, wherein said multiple electrodes are wirelessly connected to said processor.

166. The system according to claim 162, wherein said inflatable balloon is composed of an elastic polymer.

167. The system according to claim 162, wherein said inflatable balloon has a structure mimicking the structure of the stomach.

168. The system according to claim 162, wherein said multiple electrodes each have a shape selected from a group consisting of: a circular shape; an elliptic shape; and a disc shape, such that no damage is caused to said stomach wall when said multiple electrodes are in contact with the stomach wall.

169. The system according to claim 162, configured for stimulating the stomach wall by applying electrical pulses through said multiple electrodes, so as to alleviate symptoms of a condition selected from the group consisting of: gastroparesis, dyspepsia, gastroesophageal reflux disease (GERD), obesity, bulimia and eating disorders.

170. The system according to claim 162, wherein said balloon is embedded within a scaffolding shield.

171. The system according to claim 170, wherein said scaffolding shield is made of a material selected from a group consisting of: a polymer, a metal, an alloy, and a memory shape alloy.

172. A method for detecting electrical activity of the stomach, the method comprising:
inserting a deflated balloon into the stomach, said balloon having multiple electrodes mounted on its external surface and arranged as a mesh;
inflating said balloon through a tube connected to a proximal end of said balloon, such that said multiple electrodes are brought into substantially immobile contact with the stomach wall;
receiving, at a processor, a signal from said multiple electrodes, the signal corresponding to electrical activity emanating from said stomach wall and sensed by said multiple electrodes; and
translating the signal, by said processor, into an electrical activity map of the stomach.

173. The method according to claim 172, wherein said electrical activity map is selected from the group consisting of: a three-dimensional isopotential map and a two-dimensional isopotential map.

174. The method according to claim 172, further comprising detecting Gastroparesis based on the electrical activity map of the stomach.

175. The method according to claim 174, further comprising measuring the Gastroparesis based on the electrical activity map of the stomach.

176. The method according to claim 172, wherein said multiple electrodes are physically connected to said processor.

177. The method according to claim 172, wherein said multiple electrodes are wirelessly connected to said processor.

178. The method according to claim 172, wherein said balloon is composed of an elastic polymer.

179. The method according to claim 172, wherein said balloon has a structure mimicking the structure of the stomach.

180. The method according to claim 172, further comprising stimulating the stomach wall by applying electrical pulses through said multiple electrodes, so as to alleviate symptoms of a condition selected from the group consisting of: gastroparesis, dyspepsia, gastroesophageal reflux disease (GERD), obesity, bulimia and eating disorders.

181. The method according to claim 172, further comprising tuning a gastric pacemaker based on the electrical activity map of the stomach.
